(19) United States
(12) Patent Application Publication
(10) Pub. No.: US 2023/0404031 A1
(43) Pub. Date: Dec. 21, 2023

(54) REUSABLE EAR TAG

(71) Applicant: Movella Inc., Henderson, NV (US)

(72) Inventors: Yu-Chia CHANG, Henderson, NV (US); Cheng LUCKY, Henderson, NV (US); Cheng Pin HUANG, Henderson, NV (US)

(21) Appl. No.: 18/332,556

(22) Filed: Jun. 9, 2023

Related U.S. Application Data
(60) Provisional application No. 63/351,155, filed on Jun. 10, 2022.

(51) Int. Cl. A01K 11/00; A61D 13/00; A01K 29/00

(52) U.S. Cl. CPC A01K 11/004; A01K 29/005; A61D 13/00

(57) ABSTRACT

A biometric monitoring device for an animal ear includes a male portion on a first side of the ear including a base having an upper surface towards the first side including openings and a protrusion having an upper portion for penetrating from the first side a second side of the ear, and a female portion for the second side of the ear including a housing having a bottom surface towards the second side of the ear including an internal opening configured to receive the upper portion of the protrusion, a retention portion configured to releasably retain the upper portion of the protrusion, a plurality of electronic components including a battery, a processor, and a temperature sensor, and a thermally conductive ring disposed upon on the bottom surface configured to be disposed against the second side, and wherein the thermally conductive ring is thermally coupled to the temperature sensor.

### CROSS REFERENCE TO RELATED CASES

The present application is a non-provisional of and claims priority to U.S. Pat. App. No. 63/351,155 filed Jun. 10, 1922. That application is incorporated by reference herein for all purposes.

### BACKGROUND OF THE INVENTION

The present invention relates generally to apparatus for monitoring of animal health. More specifically, the present invention relates to devices capable of short-term monitoring of animals, such as livestock. Embodiments are reusable and have reduced discomfort.

Maintaining the health of animals is a major concern in the livestock industry. Previous studies have described the negative effects of external factors upon the health of animals. For example, high environmental temperature and humidity are often associated with a higher risk of disease, dehydration, weight loss, changes in behavior, reduction in food consumption, changes in blood indicators, and the like, in animals. As each animal is subject to different environmental conditions, the remote monitoring of each animal is desired.

In light of the above, what is desired are improved animal tracking tags without the drawbacks described above.

### BRIEF SUMMARY OF THE INVENTION

Embodiments of the present invention disclose an electronic animal tracker design that is less invasive, is rugged, is more reliable, and provides higher consistency readings. Some of the disclosed embodiments provide an improved ear tag having a male stud and a female stud, that provides a secure and stable fit upon an animal's ear, or the like.

In some embodiments, a new ear tag may include a housing design including carbon fiber material or other lightweight material. A male portion may include one or more thin protrusions (e.g. needles) to penetrate the animal's ear. A female portion may include one or more retention portions, e.g. indentations, that receive the protrusions and constrain the movement of the male portion away from the female portion. The female portion may also include a plurality of electronic components including a processor, a memory, a short-range communications capability, a temperature sensor, and the like. Additionally, the female portion may include a thermally conductive region (sometimes in a ring-type shape) that is pressed against the animal's ear and that thermally conducts heat from the ear to the temperature sensor.

According to one aspect, a biometric monitoring device for an ear of an animal is disclosed. One device includes a male portion configured to be disposed on a first side of the ear having a base having an upper surface configured to be disposed towards the first side of the ear, wherein the upper surface includes a plurality of openings, and at least one protrusion disposed upon the upper surface of the base, wherein an upper portion is configured to be disposed through the ear from the first side of the ear to a second side of the ear. An apparatus may include a female portion configured to be disposed on a second side of the ear having a housing having a lower surface configured to be disposed towards a second side of the ear, an internal opening in the lower surface of the housing, wherein the internal opening is configured to receive the upper portion of the protrusion, a retention portion disposed within the housing, wherein the retention portion is configured to releasably retain the upper portion of the protrusion when the first protrusion is disposed through the internal opening, wherein the male portion is secured relatively to the female portion when the retention portion retains the upper portion of the protrusion, a plurality of electronic components disposed within the housing, including a battery, a processor, and a temperature sensor, and a thermally conductive ring disposed upon on the lower surface of the housing, wherein the ring is configured to be disposed against the second side of the ear and wherein the thermally conductive ring is thermally coupled to the temperature sensor.

### DETAILED DESCRIPTION

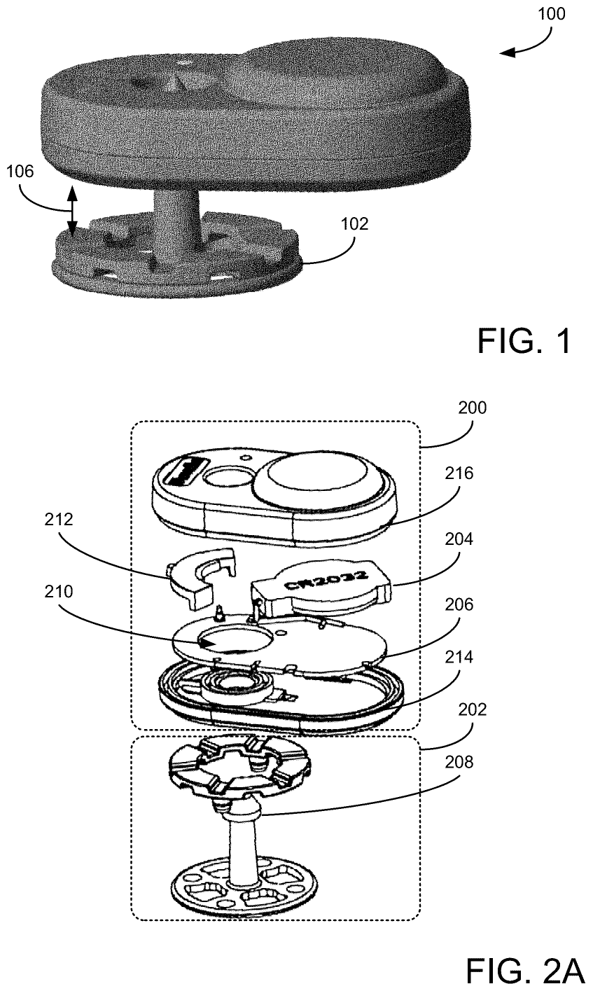

FIG. 1 illustrates various embodiments of the present invention. In some embodiments, the ear tag 100 includes two main portions, a male portion 102 and a female (larger) portion 104. An assembled ear tag is illustrated where the ear of the animal is disposed between 106 these two portions. The female portion 104 includes electronic components including a temperature sensor that is held in thermal contact against the animal's ear.

FIGS. 2A and B illustrate additional embodiments. More particularly, FIGS. 2A and B illustrate exploded views of ear tags including female portions 200 and male portions 202 according to various embodiments. Illustrated within the female portions 200 are a power source, e.g. CR2032 battery 204, a circuit board 206 having a number of electronic components, as will be discussed below, and the like.

In this example, the larger structure is a female portion 200, and the lower disk-shaped portion is a male portion 202. As can be seen, male portion 202 includes at least one retention protrusion (e.g. pointed spike) 208 extending upwards.

In various embodiments, female portion 200 includes a cavity 210 with a retention portion/mechanism 212 configured to receive the retention needle 208 from male portion 202 and configured to selectively retain and release retention needle 208. In some embodiments, the retention mechanisms may include semi-circular latch that may selectively secure and release the tip (e.g. 208) of the male portion 202 when desired, e.g. via a physical release, via a magnet release, or the like. In other embodiments, other types of retention mechanisms, such as a gasket of material, are envisioned. In some cases, the material may be rubber, silicone, plastic, or the like. This material may be used to retain the tip of the male portion, yet still provide some give so that the distance between the male portion 202 and female portion 200 may vary. In some examples, they may be stretched apart and separated by a larger distance if there is separating stress placed between them, and the portions may resume a relative distance in absence of the separating stress. Accordingly, the material may provide a cushioning effect to the animal's ear. In some examples, a rubber gasket may also be provided to reduce the intrusion of humidity or water into the female portion 200.

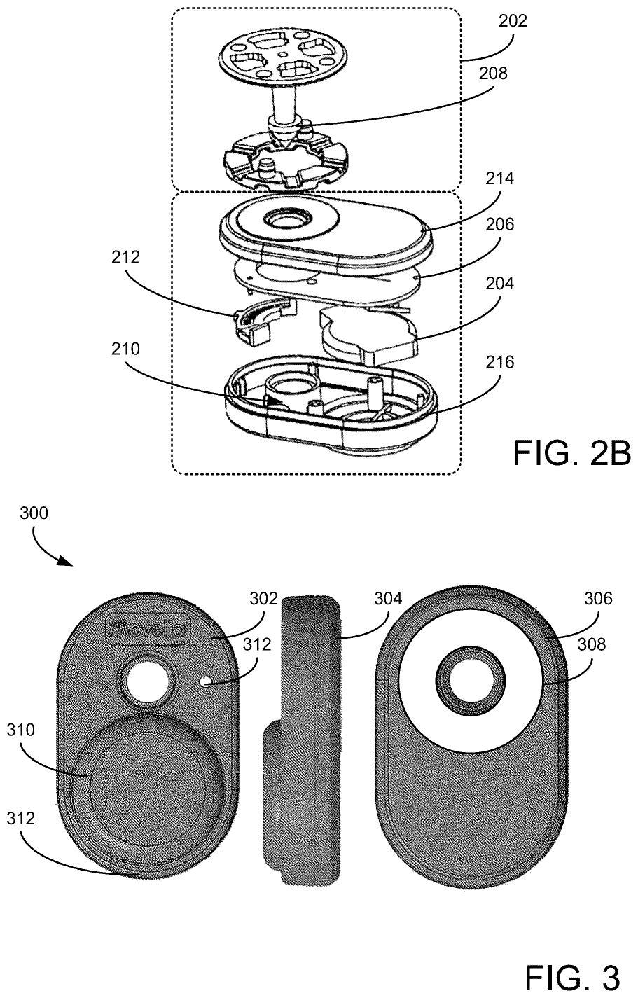

FIG. 3 illustrates additional embodiments of a female portion 300. Specifically, a front 302, side 304 and back 306 views are shown.

As illustrated, the shape of the female portion 300 may be ovoid in appearance and with reduced sharp exposed edges to reduce the potential snagging of the female portion. Accordingly, it is expected that it will be more difficult for the ear tag to be caught and dislodged from the animal.

In various embodiments, female portion 300 may include an interior cavity (e.g. 210) including electronic components such as a battery, a temperature sensor, and a thermally conductive structure (ring 308). In some embodiments, additional electronic components may include one or more of the following: a microcontroller, an accelerometer, a gyroscope, a magnetometer, a pressure sensor, a GPU unit, a short-range communications (UWB, Wi-Fi, Bluetooth, NFC, RFID) hardware, or the like.

In some embodiments, the conductive structure 308 is configured to be disposed against the skin of the ear of the animal. By having a solid contact, the temperature of the animal may be more accurately measured. In some embodiments, the thermally conductive structure 308 is thermally coupled to the temperature sensor (e.g. a thermistor, a NTC or PTC device, or the like).

In various embodiments, an closable opening 310 (e.g. a screw cap) in the female portion 300 may be opened for replacement of battery or the like. In some embodiments, an upper portion (e.g. 214) and a lower portion (e.g. 216) that can be opened for battery replacement and snapped tightly with respect to each other, or the like, to form a cavity (e.g. 210) that is protected from water, dust or dirt, and the elements. As is also illustrated, an LED 312 may provide visual output and a magnetic switch 314 may be located on various portions of female portion 300 that enables switching on or off of functionality, e.g. temperature monitoring.

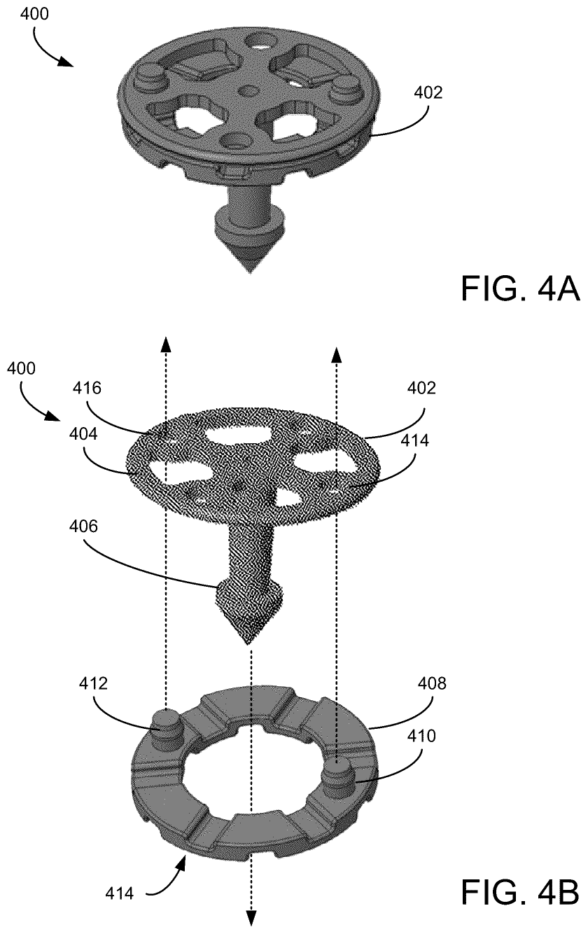

FIGS. 4A and B illustrate additional views of a male portion and components. As illustrated, in some embodiments, the male portion 400 may include two portions, a portion 402 including base 404 and a pointed protrusion 406, and an interface ring 408. The interface ring 408 is configured to engage with the base 404 and is secured to the base via one or more blunt circular upward protrusions 410 and 412, as illustrated. These blunt protrusions 410 and 412 may engage one or more holes 414 and 416 on the base 404 to form the assembled male portion shown to in FIG. 4A. In other embodiments, interface ring 408 may not be fixed relative to base 404, but may rotate.

In various embodiments, the bottom face 414 of the interface ring 408 (in the direction of the male protrusion) is configured to be disposed against the ear of the animal. As can be seen, the interface ring includes gaps so that the male portion 400 does not make full contact with the animal's ear. Although there is an increase in pressure on the remaining portions of interface ring 408, the interface ring 408 is designed to have net pressure for contact portions less than 50 mmHg. This low pressure is believed to be well within the acceptable amount of pressure to not cause appreciable injury to the animal's ears. Other designs for an interface ring 408 may have net pressures that exceed this amount of pressure, and may cause ulceration, or the like.

In various embodiments, additional openings in the base structure 404 allows air to flow to the site on the ear the ear tag penetrates. This air flow is believed to promote healing of the ear and reduce the risk of infection of the wound site.

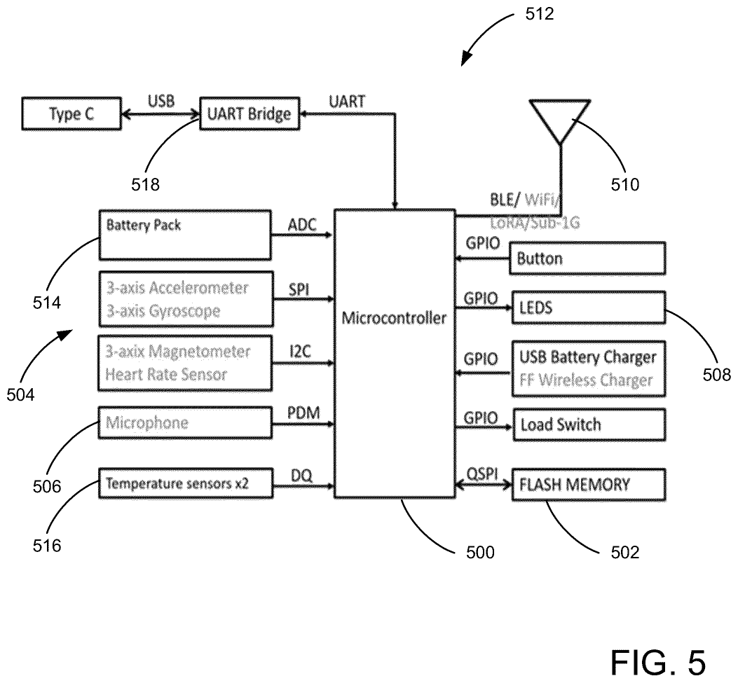

FIG. 5 illustrates a block diagram of an embodiment. More specifically, FIG. 5 illustrates a block diagram of various embodiments of an electrical devices that may be incorporated into a female portion or a male portion of an ear tag, or the like.

In some embodiments, a typical device 512 may include a processor or microcontroller 500, a memory 502, physical perturbation sensors (e.g. accelerometer, gyroscope, pressure sensor) 504, audio/video sensors 506, sensory output devices 508 (e.g. lights, speakers), short-range transceivers 510 (Bluetooth, ultrawide band (UWB), near field communication (NFC), radio frequency identification (RFID), biosensors (e.g. heart rate), an internal power supply 514, temperature sensors 516, a wired interface 518, and the like.

In some configurations, the processor 500 may be an ARM Cortex processor with Bluetooth LE RF, although other low-power processors may also be used. As illustrated above, microcontroller 800 may include numerous functionality such as a transceiver, temperature sensors, serial communications functionality (e.g. UART), comparators, and the like. In some embodiments, a superset or subset of the hardware and software functionality described herein may be used.

In various embodiments, data processing algorithms may be included in the flash memory. Examples of algorithms may include: gait analysis, heart rate variability, energy exertion, respiration rate, sound classification, direction or heading determination, steps, joint angle, pose detection, estrus detection, mortality reduction, and the like. By having such algorithms being performed on the monitoring device, i.e. on the edge, the amount of data output by the monitoring device to an external computing system can be greatly decreased.

Further embodiments can be envisioned to one of ordinary skill in the art after reading this disclosure. For example, in some embodiments, these biometric monitoring devices may be affixed to one or more animals within a herd, flock, or the like. Data regarding each of the monitored animals may then be wireless provided to a local transceiver using Bluetooth, RF, or the like, as described above. The local transceiver then uploads the animal data to a monitoring service (e.g. a physical or virtual server) that processes the animal data. Using artificial intelligence, e.g. machine learning algorithms, the monitoring service may then monitor each individual animal based upon its own specific physiology, monitor the weather conditions, and determine whether the individual health status of the individual animal. Alternative or additionally, the individual data from the group of animals may be processed, also using AI techniques, to determine the health of the entire group. In response to the health data, the monitoring service may determine a list of actions the farmer is required/desired to do for the individual animal or the group. For example, if an individual animal is running a temperature while other animals are not, the individual may be flagged as unwell. The farmer may also be instructed to separate the individual animal from the group and have a veterinarian look over the animal. As additional examples, if all animals in a group are running a temperature and the monitoring service determines that there is a heat wave (processing weather data), the farmer may be instructed to bring the animals into a covered or shaded location, give the animals more water, provide a mister, not to transport the animals or the like. Further, in this example, if the monitoring service determines that there is no heat wave, the animals may all be sick, and the monitoring service may instruct the farmer to consult a veterinarian. In other embodiments, the monitoring service may also receive and process other types of data to help determine the health of animals, such as the amount of movement of the animal, the amount of sleep, the type of feed provided, the feeding times, the amount of feed per head provided, the other types of animals on the farm, the amount of space per animal, the types of medications used, the types of crops and fertilizers used in fields adjacent to the farmer's land, the weather (e.g. precipitation, barometric pressure profiles, wind speed profiles, air temperature profiles, the amount of sunshine, the air quality and the like), the amount of local noise (e.g. car traffic, air traffic, construction), the time of year, and the like. Using such broad ranges of data and machine learning algorithms, using embodiments of the present invention, it is expected that the farmer will be provided with health insights to animals that the farmer cannot determine themselves.

The block diagrams of the architecture and flow charts are grouped for ease of understanding. However, it should be understood that combinations of blocks, additions of new blocks, re-arrangement of blocks, and the like are contemplated in alternative embodiments of the present invention. The specification and drawings are, accordingly, to be regarded in an illustrative rather than a restrictive sense. It will, however, be evident that various modifications and changes may be made thereunto without departing from the broader spirit and scope of the invention as set forth in the claims.

We claim:

1. A biometric monitoring device for an ear of an animal comprises:
a male portion configured to be disposed on a first side of the ear comprising:
a base having an upper surface configured to be disposed towards the first side of the ear, wherein the upper surface includes a plurality of openings; and
at least one protrusion disposed upon the upper surface of the base, wherein an upper portion is configured to be disposed through the ear from the first side of the ear to a second side of the ear; and
a female portion configured to be disposed on a second side of the ear comprising:
a housing having a lower surface configured to be disposed towards a second side of the ear;
an internal opening in the lower surface of the housing, wherein the internal opening is configured to receive the upper portion of the protrusion;
a retention portion disposed within the housing, wherein the retention portion is configured to releasably retain the upper portion of the protrusion when the first protrusion is disposed through the internal opening, wherein the male portion is secured relatively to the female portion when the retention portion retains the upper portion of the protrusion;
a plurality of electronic components disposed within the housing, including a battery, a processor, and a temperature sensor; and
a thermally conductive ring disposed upon on the lower surface of the housing, wherein the ring is configured to be disposed against the second side of the ear and wherein the thermally conductive ring is thermally coupled to the temperature sensor.

2. The device of claim 1
wherein the temperature sensor is characterized by an electronic characteristic in response to a temperature of the temperature sensor; and
wherein the electronic characteristic is selected from a group consisting of: an electronic resistance, a voltage.

3. The device of claim 1 wherein the housing comprises a material selected from a group consisting of: plastic, metal, silicone.

4. The device of claim 1 wherein the plurality of electronic components includes a short-range communications circuits selected from a group consisting of: near-field communication (NFC), ultrawide band (UWB), Radio Frequency Identification (RFID), and Bluetooth (BLE).

5. The device of claim 1 wherein the plurality of electronic components further includes an electronic component selected from the group consisting of: an accelerometer, a gyroscope, a humidity sensor, an IR sensor, a magnetometer, a heart rate sensor, an electrocardiogram (ECG) sensor, a photoplethysmography (PPG) sensor, a microphone and a pressure sensor.

6. The device of claim 1 wherein a material for the thermally conductive ring is selected from a group consisting of: silver or silver alloy, copper or copper alloy, aluminum compound, gold, diamond, silicon nitride, silicon carbide, graphite, and graphene.

7. The device of claim 1
wherein the plurality of electronic components includes a magnetic switch configured to be activated, and
wherein the short-range communications circuits is configured to output data to a remote computing device in response to an activation of the magnetic switch.

8. The device of claim 1
wherein the device further comprises a spacer portion configured to be disposed between the first side of the ear and the upper surface of the base,
wherein the spacer portion includes a first side,
wherein first portions of the first side are configured to be disposed against the first side of the ear,
wherein second portions of the first side are configured to be disposed a distance away from the first side of the ear; and
wherein the second portions are configured to allow air to flow between the spacer portion and the first side of the ear.

9. The device of claim 8 wherein the spacer portion is configured to rotate relative to the at least one protrusion.

10. The device of claim 8
wherein the spacer portion or the male portion includes a protrusion; and
wherein the male portion or the spacer portion comprises an opening, wherein the opening is configured to receive and physically retain the protrusion of the spacer portion or the male portion.

11. A biometric monitoring device for an ear of an animal comprises:
a male portion configured to be disposed on a first side of the ear comprising:
a base having an upper surface configured to be disposed towards the first side of the ear, wherein the upper surface includes a plurality of openings;
a spacer configured to be disposed between the first side of the ear and the upper surface of the base, wherein only a portion of the spacer contacts the first side of the ear, wherein the portion of the spacer is substantially flat; and
at least one protrusion disposed upon the upper surface of the base extending through the spacer, wherein an upper portion is configured to be disposed through the ear from the first side of the ear to a second side of the ear; and
a female portion configured to be disposed on a second side of the ear comprising:
a housing having a lower surface configured to be disposed towards a second side of the ear;
an internal opening in the lower surface of the housing, wherein the internal opening is configured to receive the upper portion of the protrusion;
a retention portion disposed within the housing, wherein the retention portion is configured to repeatedly retain the upper portion of the protrusion when the first protrusion is disposed through the internal opening, wherein the male portion is secured relatively to the female portion when the retention portion retains the upper portion of the protrusion, wherein the retention portion comprises a release configured to repeatedly release the upper portion of the protrusion from the retention portion;
a plurality of electronic components disposed within the housing, including a battery, a processor, and a temperature sensor; and
a thermally conductive ring disposed upon on the lower surface of the housing, wherein the ring is configured to be disposed against the second side of the ear and wherein the thermally conductive ring is thermally coupled to the temperature sensor.

12. The device of claim 11
wherein the temperature sensor is characterized by an electronic characteristic in response to a temperature of the temperature sensor; and
wherein the electronic characteristic is selected from a group consisting of: an electronic resistance, a voltage.

13. The device of claim 11 wherein the housing comprises a material selected from a group consisting of: plastic, metal, silicone.

14. The device of claim 1 wherein the plurality of electronic components includes a short-range communications circuits selected from a group consisting of: near-field communication (NFC), ultrawide band (UWB), Radio Frequency Identification (RFID), and Bluetooth (BLE).

15. The device of claim 11 wherein the plurality of electronic components further includes an electronic component selected from the group consisting of: an accelerometer, a gyroscope, a humidity sensor, an IR sensor, a magnetometer, a heart rate sensor, an electrocardiogram (ECG) sensor, a photoplethysmography (PPG) sensor, a microphone and a pressure sensor.

16. The device of claim 11 wherein a material for the thermally conductive ring is selected from a group consisting of: silver or silver alloy, copper or copper alloy, aluminum compound, gold, diamond, silicon nitride, silicon carbide, graphite, and graphene.

17. The device of claim 11
wherein the plurality of electronic components includes a magnetic switch configured to be activated, and
wherein the short-range communications circuits is configured to output data to a remote computing device in response to an activation of the magnetic switch.

18. The device of claim 11 wherein the device includes at least one light emitting diode (LED) configured to provide a visual output.

19. The device of claim 11 wherein the spacer portion is configured to rotate relative to the base.

20. The device of claim 11 wherein the spacer portion is configured to be stationary relative to the base.
